(19) United States
(12) Patent Application Publication
(10) Pub. No.: US 2007/0191881 A1
(43) Pub. Date: Aug. 16, 2007

(54) Tourniquet

(76) Inventors: Shai Amisar, Tel-Aviv (IL); Ronem Radomski, Haifa (IL)

Correspondence Address: Shiboleth Yisraeli Roberts Zisman & Co, 350 Fifth Avenue, 60th Floor, New York, NY, 10118, US

(21) Appl. No.: 10/594,632

(22) PCT Filed:
(86) PCT No.: PCT/IL05/00353

(30) Foreign Application Priority Data
Mar. 29, 2004 (IL) 161159

(51) Int. Cl. A61B 17/00

(52) U.S. Cl. 606/203

(57) ABSTRACT

A self-regulating tourniquet has a pressure source, a pressure applicator apparatus associated with the pressure source and operative to apply pressure therefrom to a limb and a pressure regulator associated with the pressure source and the pressure applicator operative to restrict the pressure transferred from the pressure source to the limb to a designated maximum pressure applied to the limb. The pressure source and the pressure applicator may be separate modules that are adapted to be engaged by connecting means and establish gas passage from the pressure source to the pressure applicator.

### FIELD OF THE INVENTION

The present invention relates to tourniquets generally, and to field tourniquets in particular.

### BACKGROUND OF THE INVENTION

A tourniquet is a device that is intended to stop bleeding from traumatic wounds in the arms and legs. Essentially, tourniquets may be divided into two main categories, namely, those for use in the field, and those for use during surgical procedures in an operating room. Typical types of field tourniquets work by the application of external pressure on a bleeding limb, by use of flexible bands which may or may not be elastic. A disadvantage common to all known field tourniquets is the application of non-regulated pressure (either too much pressure so as to cause unnecessary harm to the subject, or too little pressure so as to be ineffective). A further disadvantage of known field tourniquets is that they cannot normally be applied by the wounded person to himself, and require the presence of a second person for use. While pneumatically operated tourniquets for hospital use exist, they require connection to a ready supply of pressurize air or electricity, and are thus totally unsuitable for use in the field. Among known art are the following US patents: U.S. Pat. No. 4,479,494 entitled Adaptive pneumatic tourniquet; U.S. Pat. No. 4,516,576 entitled Tourniquet strap or band for restricting blood flow, especially for taking blood samples; U.S. Pat. No. 4,520,819 entitled Tourniquet with differential pressure occlusion detector; U.S. Pat. No. 4,520,820 entitled Automatic tourniquet with improved pressure resolution; U.S. Pat. No. 4,548,198 entitled Automatic tourniquet; U.S. Pat. No. 4,671,290 entitled Automatic tourniquet; U.S. Pat. No. 5,048,536 entitled Tourniquet for regulating applied pressures; U.S. Pat. No. 5,181,522 entitled Tourniquet for sensing and regulation of applied pressure; U.S. Pat. No. 5,254,087 entitled Tourniquet apparatus for intravenous regional anesthesia; U.S. Pat. No. 5,439,477 entitled Tourniquet apparatus for applying minimum effective pressure; U.S. Pat. No. 5,540,714 entitled Disposable tourniquet; U.S. Pat. No. 5,556,415 entitled Physiologic tourniquet for intravenous regional anesthesia; U.S. Pat. No. 5,584,853 entitled Tourniquet cuff apparatus; U.S. Pat. No. 5,607,447 entitled Physiologic tourniquet; U.S. Pat. No. 5,649,954 entitled Tourniquet cuff system; U.S. Pat. No. 5,741,295 entitled Overlapping tourniquet cuff system; U.S. Pat. No. 5,842,996 entitled Automatic tourniquet system; U.S. Pat. No. 5,855,589 entitled Physiologic tourniquet for intravenous regional anesthesia; U.S. Pat. No. 5,911,735 entitled Time-limited Physiologic tourniquet; U.S. Pat. No. 5,931,853 entitled Physiologic tourniquet with safety circuit; U.S. Pat. No. 6,213,939 entitled Hazard monitor for surgical tourniquet systems; U.S. Pat. No. 6,299,629 entitled Automatic tourniquet system; U.S. Pat. No. 6,589,268 entitled Hazard monitor for surgical tourniquet systems; and U.S. Pat. No. 6,682,547 entitled Tourniquet cuff with identification apparatus.

### SUMMARY OF THE INVENTION

It is thus an aim of the present invention to provide a self-regulating tourniquet which accurately regulates the pressure applied to a wound, which is simple to use, may be self-applied by a subject, and which is fully portable so as to be suitable for use in the field.

### DETAILED DESCRIPTION OF THE DRAWINGS

The present invention will be more fully understood and appreciated form the following detailed description, taken in conjunction with the drawings, in which:

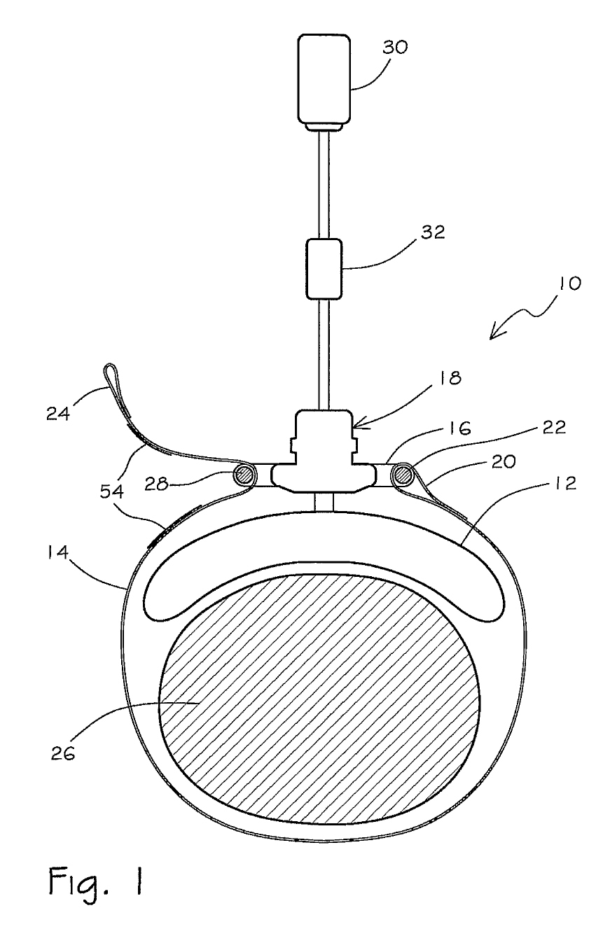

FIG. 1 is a schematic representation of a self-regulating inflatable tourniquet constructed and operative in accordance with a preferred embodiment of the present invention;

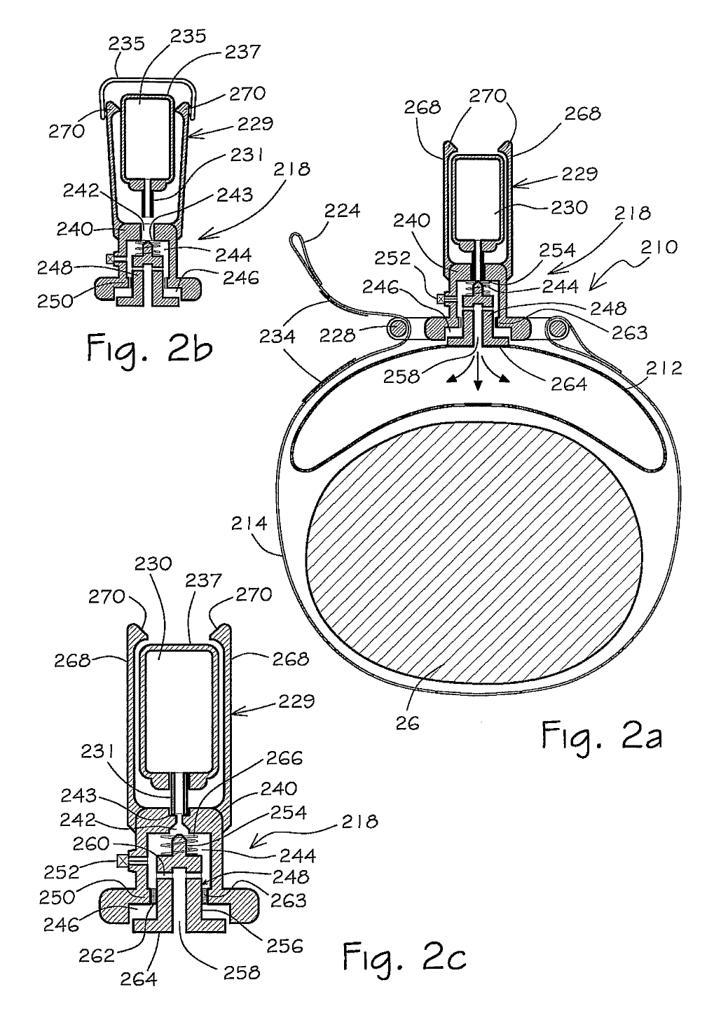

FIG. 2A is a cross-sectional view of a self-regulating inflatable tourniquet constructed and operative in accordance with a first embodiment of the present invention, during application;

FIGS. 2B and 2C are enlarged cross-sectional views of the operating system of the tourniquet seen in FIG. 2A, in positions prior to and during application, respectively;

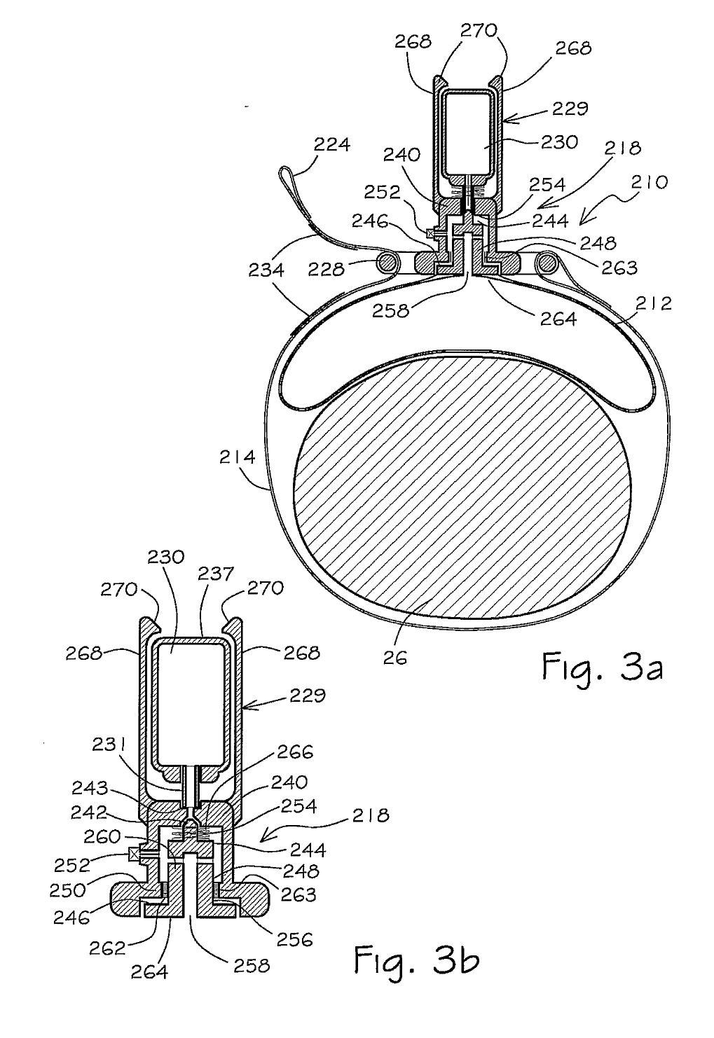

FIG. 3A is a view similar to that of FIG. 2A, but showing the self-regulating inflatable tourniquet in a position subsequent to application;

FIG. 3B is an enlarged cross-sectional view of the operating system of the tourniquet seen in FIG. 3A;

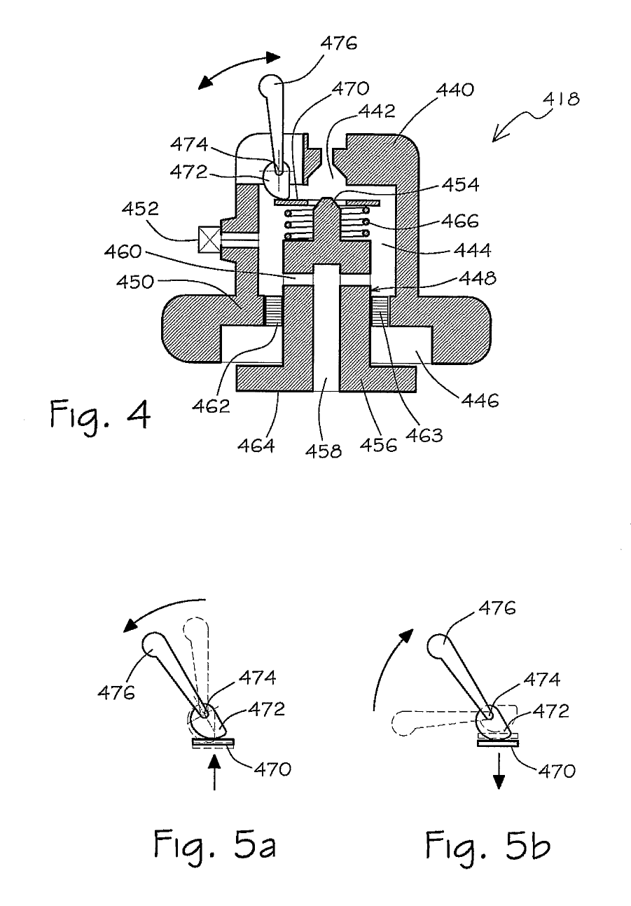

FIG. 4 is an enlarged cross-sectional view of an adjustable pressure regulator employed in the self-regulating inflatable tourniquet of the resent invention;

FIGS. 5A and 5B are illustrations of a selector employed in the adjustable pressure regulator of FIG. 4, seen in positions of increasing pressure and decreasing pressure adjustment, respectively;

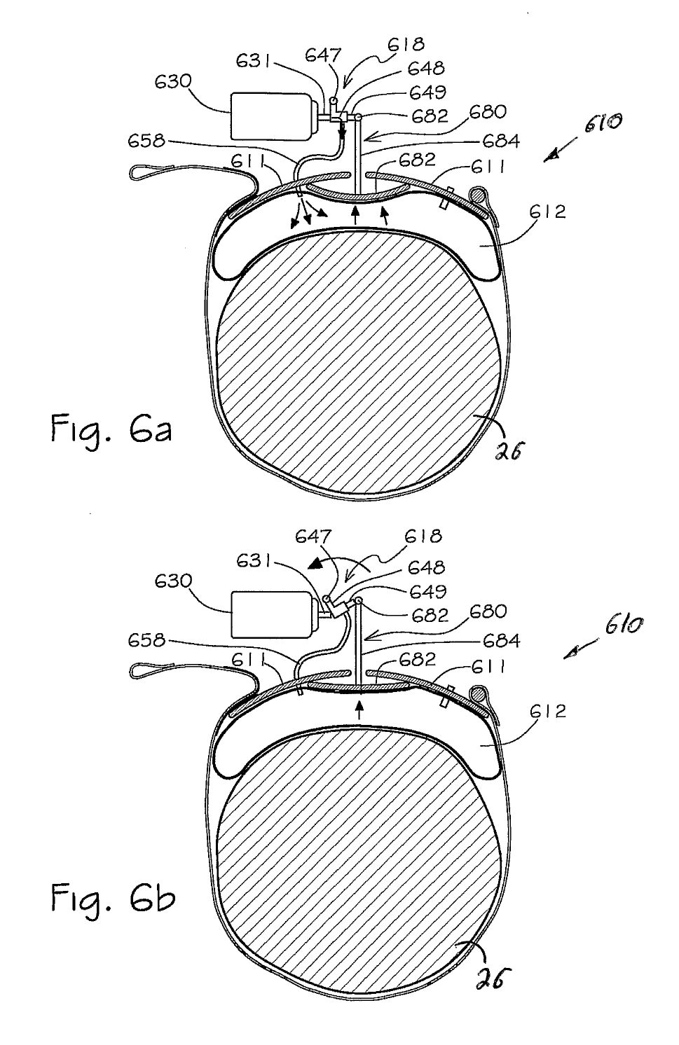

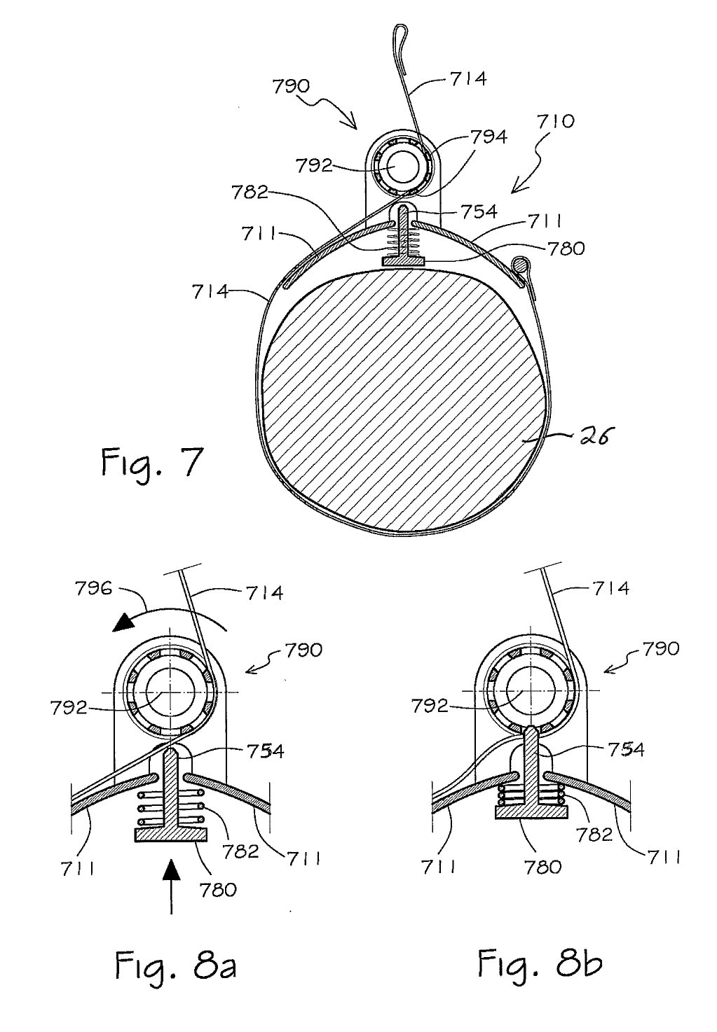

FIGS. 6A and 6B are cross-sectional illustrations of a self-regulating inflatable tourniquet constructed and operative in accordance with a further embodiment of the present invention, during and after application, respectively;

FIG. 7 is a cross-sectional view of a self-regulating non-inflatable tourniquet constructed and operative in accordance with yet a further embodiment of the present invention, during application; and

FIGS. 8A and 8B are enlarged views of an automatic pressure regulator seen in FIG. 7, during application of the tourniquet and after application thereof, respectively.

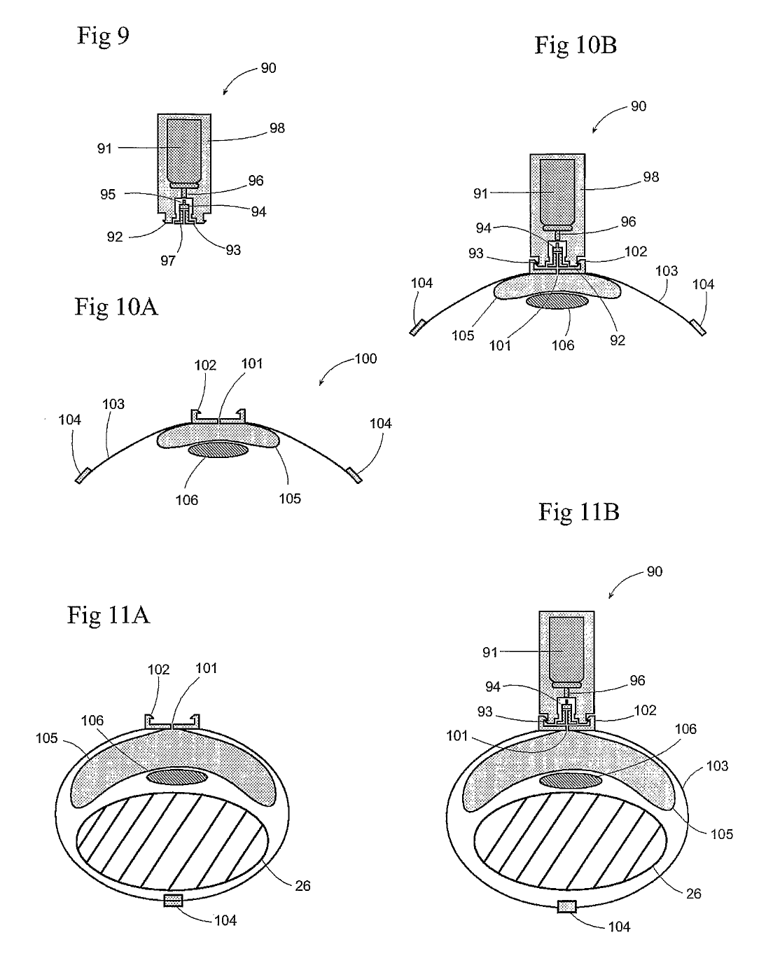

FIG. 9 shows a pressure module of a modular self-regulating tourniquet;

FIGS. 10A and 10B shows the tourniquet module of modular self-regulating tourniquet with and without the pressure module, wherein the contra member is opened;

FIGS. 11A and 11B show the tourniquet module of modular self-regulating tourniquet with and without the pressure module, wherein the contra member is closed and the pressure member is in an inflated state;

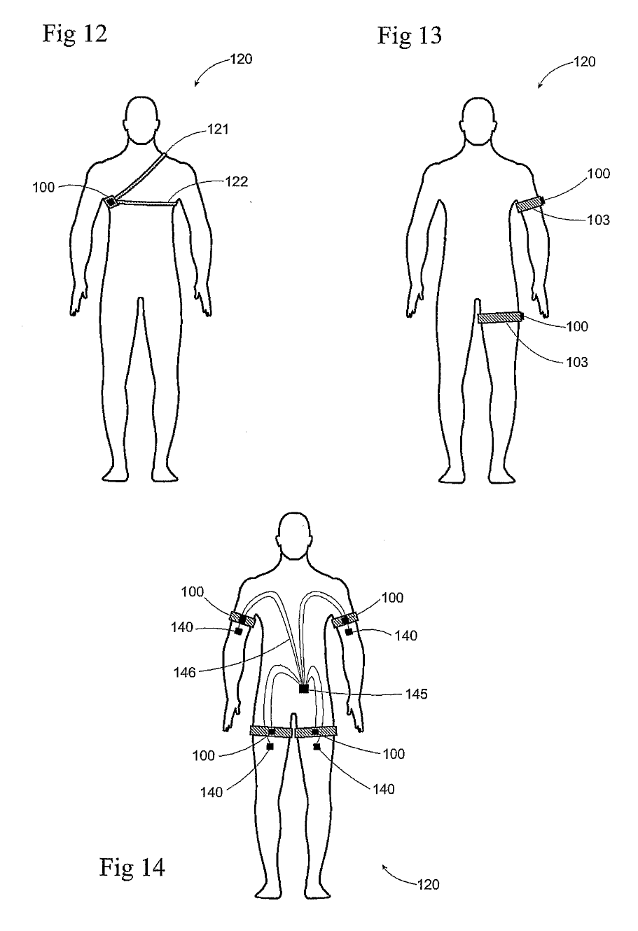

FIG. 12 illustrates an implementation of the modular self-regulating tourniquet of the invention adapted to be used on a section of the torso to press a pressure point;

FIG. 13 illustrates applying the modular self-regulating tourniquet on limbs of a human subject; and

FIG. 14 shows an embodiment of the modular self-regulating tourniquet wherein the operation of a number of modular self-regulating tourniquets, which may be embedded in wearable articles such as uniforms, is controlled by a controller.

### DETAILED DESCRIPTION OF THE INVENTION

As described above, it is an aim of the present invention to provide a self-regulating tourniquet which accurately regulates the pressure applied to a wound, which is simple to use, may be self-applied by a subject, and which is fully portable so as to be suitable for use in the field.

In the present application, a number of different types of self-regulating tourniquet are exemplified. Specifically, one type is a self-regulating inflatable tourniquet, such as exemplified in FIGS. 1-6B and 9-14, while a non-inflatable type is exemplified in FIGS. 7-8B.

Referring now initially to FIG. 1, there is illustrated a self-regulating inflatable tourniquet, referenced generally 10, which includes an inflatable pressure member 12 and a contra member 14, which cooperate so as to form pressure applicator apparatus.

Pressure member 12 and contra member 14 are connected via a yoke 16. Pressure member 12 is floatingly supported via a pressure regulator 18 to which yoke 16 is fixed. Contra member 14, however, which preferably is an elongate, flexible, belt like member, is attached at a first end 20 to a first side 22 of yoke 16, and has a second, free end 24, which is looped around a limb 26 to which the tourniquet is to be applied, and threaded through a second side 28 of yoke 16. Subsequently, the contra member 14 is pulled tight, and fastened down as via Velcro® or other equivalent fastening system, indicated at 54.

Inflatable pressure member 12 is adapted to be pressurized by a pressurized gas or liquid pressure source 30, the pressure from which may be restricted by a suitable valve 32, so as not to over-pressurize the inflatable pressure member. Pressure regulator 18 is disposed between valve 32 and inflatable pressure member 12, and is operative to restrict the pressure transferred from pressure source 30 to limb 26, to a desired maximum pressure applied to the subject limb. Various examples of pressure regulator 18 are shown and described hereinbelow, in detail.

Reference is now made to FIG. 2A, in which is illustrated a specific embodiment of the self-regulating inflatable tourniquet shown and described above in conjunction with FIG. 1, in accordance with a first preferred embodiment of the present invention. Where appropriate, all parts of the illustrated tourniquet that have counterparts described above in conjunction with FIG. 1, are not specifically described again herein, and are denoted by similar reference numerals, but with the addition of the prefix “2”.

Referring now also to FIGS. 2B and 2C, pressure regulator 218 is seen to have an inverted generally cup-shaped housing 240, onto which is mounted a support 229 for pressure source 230. Housing 240 has a pressure inlet 242, through which a pressurized fluid is received; an inner chamber 244; an outer chamber 246 for seating a hollow pressure transfer element 248 through which pressurized fluid is transferred to inflatable pressure member 212; and a waist portion 250 disposed between inner and outer chambers 244 and 246. An excess pressure valve 252 is also provided, in the side wall of inner chamber 244.

Pressure transfer element 248 is formed so as to include a sealing pin 254, adapted to seal pressure inlet 242 when forced thereagainst; a hollow intermediate portion 256, having a central passage 258 and pressure inlets 260 formed in the side wall thereof, and adapted for travel through an opening 262 formed by waist portion 250; and a flared portion 264, formed integrally with inflatable pressure element 212, and through which central passage 258 opens into the interior of pressure element 212. A seal 263 is provided in opening 262, thereby to prevent an escape of pressurized fluid from inner chamber 244 to outer chamber 246. A compression element, referenced 266, is mounted over sealing pin 254, thereby to determine the resistive force which must be overcome by pressure transfer element 248 in order for sealing pin 254 to be forced against pressure inlet 242 so as to seal it and thus prevent further inflation of inflatable pressure element 212, and thus prevent application of additional pressure to the limb 26.

In the present example, support 229 for pressure source 230 is seen to include resilient side portions 268 which has inward-facing teeth 270 adapted to contact the pressure source 230 so as to be forced resiliently apart by the partial insertion therebetween of the pressure source 230, as seen in FIG. 2B, and so as to apply a gripping force thereto. The length of support 229 is such that when the pressure source is held between teeth 270, a fluid dispensing nozzle 231 of pressure source 230 is not engaged with pressure inlet 242, and thus no pressurized fluid is dispensed. In accordance with a preferred embodiment of the invention, in which pressure source 230 is an aerosol or similar device, the pressurized fluid therein is dispensed by an axial depression of nozzle 231, such as would occur when depressing pressure source 230 against a shoulder portion 243.

Use of the tourniquet 210 is thus as follows:

Prior to use, tourniquet 210 is positioned by placing pressure member 212 over a required site on limb 26, and is fastened in position by looping contra member 214 about the limb, and fastening it, as shown.

A cap 235 (FIG. 2B) is then removed from pressure source support 229, and pressure source 230 is then pressed downwardly with respect to the support 229, so that nozzle 231 becomes engaged with shoulder 243, so as to initiate dispensing of the pressurized fluid from pressure source 230. The bottom end 237 of pressure source 230 becomes displaced beyond teeth 270, such that the pressure source 230 is held in fluid dispensing position thereby.

As seen in FIGS. 2A and 2C, the pressurized fluid dispensed from pressure source 230 flows through pressure inlet 242 and into inflatable pressure member 212, via inner chamber 244, pressure inlets 260, and central passage 258. As the pressure member 212 inflates against limb 26, the pressure thereagainst builds, due to the presence of contra member 214 in the illustrated, fastened position.

During this time, the pressure transfer element 248 is held in an open position with respect to the pressure inlet 242 by compression member 266, and will remain in an open position as long as the resistive force of the compression member 266 is greater than an opposing force applied thereto by pressure member 212, via pressure transfer element 248.

Referring now to FIGS. 3A and 3B, as the pressure applied to limb 26 by pressure member 212 and contra member 214 builds, however, to a magnitude sufficient to overcome the resistance of compression member 266, pressure transfer element 248 is displaced outwardly, through housing 240, until sealing pin 254 engages pressure inlet 242. At this point, the pressure within inflatable pressure member 212, and thus the pressure applied to limb 26, ceases to increase, having reached a predetermined magnitude.

Subsequently, in the event that there is a pressure loss, for example, caused by a sharp drop in temperature, the pressure transfer element 248 will be forced away from pressure inlet 242 by compression member 266, thereby permitting pressurized fluid to pass through inlet 242 so as to cause a partial re-inflation of pressure member 212, until the predetermined pressure is reached once again.

Conversely, in the event that there is a pressure increase, which may be caused, for example, by a sharp increase in temperature, any excess pressure is automatically released by excess pressure valve 252.

Referring now briefly to FIG. 4, there is illustrated a pressure regulator 418 which is generally similar to pressure regulator 218 shown and described above in conjunction with FIGS. 2A-3B, but in which it is possible to adjust the pressure to be applied by the tourniquet device of the present invention. All components and portions forming part of adjustable pressure regulator 418, and having counterparts in pressure regulator 218, and denoted herein with similar reference numerals, and are not specifically shown and described again herein.

It is clear that the magnitude of the pressure, referred to herein also as “designated pressure”, which is applied by the tourniquet of the present embodiment of the invention, is dictated by the resistive force of the compression element 266, and the travel of pressure transfer element 248 before engagement of the pressure inlet 242 by sealing pin 254.

It will be appreciated by persons skilled in the art, that in some cases it is desirable to be able to adjust the designated pressure. Accordingly, in accordance with the present embodiment of the invention, a camming mechanism is provided for adjusting the travel required by the pressure transfer element 248 before sealing pin 254 engages the pressure inlet 242.

Camming mechanism includes an apertured cam plate 470, mounted onto a free end of compression member 466, and a cam 472 which is mounted about a pivot axis 474. A selector lever 476 is attached to cam 472 so as to facilitate manual selection of a lower designated pressure, as illustrated in FIG. 5A, or a higher designated pressure.

Reference is now made to FIGS. 6A and 6B, in which is illustrated a further embodiment of the self-regulating inflatable tourniquet shown and described above in conjunction with FIG. 1. Where appropriate, all parts of the illustrated tourniquet that have counterparts described above in conjunction with FIG. 1, are not specifically described again herein, and are denoted by similar reference numerals, but with the addition of the prefix “6”.

Pressure regulator 618 is seen to employ a pressure transfer element 648 which is adapted to pivot about a pivot axis 647 whose location is preferably fixed with respect to the pressure source 630. Element 648 is hollow, and fluid passing thereinto from pressure source 630, is conveyed through element 648, to a flexible fluid conduit 658, which passes through a sensor positioning plate 611 (whose function is described hereinbelow) mounted onto inflatable pressure member 612, and into the interior of pressure member 612. A distal end 649 of transfer element 648, is pivotably connected to a pressure sensing member 680, at a floating pivot axis 682.

Pressure sensing member 680 is seen to have a resilient cusp element 682, which is arranged to lie against an outer surface of inflatable pressure member 612, and is confined between the pressure member 612 and positioning plates 611. The function of the positioning plates is solely to maintain cusp element 682 in pressure sensing relation with pressure member 612.

Pressure sensing member 680 further includes a rigid force transfer member 684, which affords a rigid connection between cusp element 682 and distal end 649 of transfer element 648.

In operation, after initial positioning of the tourniquet 610 on the limb 26 generally as described above, aerosol nozzle 631 is engaged with pressure transfer element 648, so as to release pressurized gas into inflatable member 612, via conduit 658. As member 612 is inflated, the increase in pressure forces pressure sensing member 680 in an outward direction, causing a flattening of resilient cusp element 682, thereby also forcing force transfer member 684 in a similarly, outward direction.

As force transfer member 684 is travels outwardly, it rotates pressure transfer element 648 in the illustrated, anti-clockwise direction, thereby causing a disengagement thereof from nozzle 631. Once complete disengagement is achieved, the flow of pressurized gas stops, so as to stabilize the tourniquet pressure at the designated pressure.

Referring now to FIG. 7, the present invention also includes, as described above, a self-regulating non-inflatable tourniquet, of which one example is shown and described herein. Where appropriate, all parts of the presently illustrated tourniquet that have counterparts described above in conjunction with FIGS. 1-6B, are not specifically described again herein, and are denoted by similar reference numerals, but with the addition of the prefix “7”.

In the present embodiment, the contra member 714 is looped about a pulley 790, and also functions as a pressure source, in conjunction with a manually applied pulling force. The pulley 790 has a typically circular locking element 792 fixable mounted thereon, against which the contra member 714 is pulled, as the tourniquet is tightened. Locking element 792 has a plurality of locking recesses 794 formed thereon, whose function is described hereinbelow.

Referring now also to FIGS. 8A and 8B, a pressure sensing member 780 is resiliently mounted on the underside of a pair of positioning plates 711, via a resilient element 782. Pressure sensing member terminates in a locking pin 754, for selectively engaging an adjacent locking recess 794.

In operation, after initial positioning of the tourniquet 710 on the limb 26 generally as described above, contra member 714 is looped through pulley 790. As it continues to be pulled, shown schematically by arrow 796 in FIG. 8A, and the pressure increases, the pressure regulator 718 is forced inwardly, so that retaining plates 711 depress resilient element 782. As thus continues, locking element 792 is forced into contact with locking pin 754, until an adjacent locking recess 794 is lockably engaged thereby, so as to trap the contra member 714 therebetween, thus stabilizing the pressure on the limb 26 at the designated pressure.

By using liquefied gas as the source of the pressure and by employing its inherent liquid/gas phase's steady-state pressure, we can use the same embodiment as in FIG. 2A without the need of a pressure regulator. When the device is activated, the liquefied gas flow into the inflatable bladder and produce a pressure inherent to its chemical composition. We will apply this physical phenomenon to regulate the pressure applied to a limb. Further more, the proximity of the bladder to the limb will regulate the temperature the gas is subjected to and will help stabilizing the pressure on the limb at the designated pressure.

FIGS. 9, 10A-10B and 11A-11B illustrate another preferred embodiment of the invention wherein the self-regulating tourniquet comprise two distinct modules, a pressure module 90 (FIG. 9), and a tourniquet module 100 (FIG. 10). The pressure module 90 consists of a housing 98 comprising a pressure source 91 disposed therein. The opening of nozzle 96 of pressure source 91 is in fluid connection with the inner chamber 95 provided in housing 98. Inner chamber 95 comprises a sealing pin 95 and a pressure transfer element 93. A male fitting 92 is provided at one end of housing 98 where an opening of inner chamber 95 is formed. Male fitting 92 is adapted to engage a respective female fitting 102 provided on the tourniquet module 100 and thereby to provide a sealed closure of inner chamber 95.

When the pressure module 90 is attached to the tourniquet module 100 the opening 97 of central passage of pressure transfer element 93 is placed in connection with aperture 101 provided in female fitting 102 of tourniquet module 100. Aperture 101 is in fluid connection with the interior of pressure member 105, such that a fluid passage from inner chamber 95 via central passage of pressure transfer element 93 and aperture 101 is obtained when pressure module 90 is attached to tourniquet module 100. Pressure member 105 is attached to the bottom side of female fitting 102 and it is adapted to be inflated by pressurized fluid that is released from pressure source 91 when the pressure module 90 is attached to the tourniquet module 100.

Pressure member 105 comprise a firm member 106 disposed at its bottom side and adapted to deliver direct pressure to a specific point on limb 26, thus causing a specific blood vessel to close, as shown in FIG. 11B. A contra element 103 comprising two flexible members is attached to the side of the female fitting 102. Fastening means 104 are provided at the free ends of the members of the contra element 103 for forming a closed loop when the tourniquet module is fastened around limb 26, as shown in FIGS. 11A-11B wherein the pressure member is shown in an inflated state.

The procedure of applying the modular tourniquet of the invention comprise fastening the members of the contra element 103 of the tourniquet module 100 around limb 26 via fastening means 104, attaching the pressure module 90 to the tourniquet module 100 via male 92 and female 102 fittings. Pressure member 105 may be inflated either manually by attaching a manual inflation device thereto (not shown in FIGS. 9, 10A-10B and 11A-11B), or automatically by attaching pressure module 90 to the tourniquet module 100 such that the inner valve of pressure module 90 is opened, thereby inflating the pressure member 105. This modular self-regulating tourniquet is simple to use, may be self-applied by a subject, and is fully portable so as to be suitable for use in the field.

Pressure module 90 is preferably made of an impact resist polymer, such as lexan, and it may be manufactured by an injection molding process. The height of pressure module 90 is generally in the range of 30-60 mm, preferably about 45 mm, its diameter is generally in the range of 15-50 mm, preferably about 25 mm, the diameter of inner chamber 95 is generally in the range of 12-40 mm, preferably about 25 mm, and the diameter of male fitting 92 is generally in the range of 10-30 mm, preferably about 20 mm. Pressure source 91 is preferably a type of gas container filled with liquefied gas (e.g., tetrafluoro ethane) in pressures of about 5 bar and volumes of about 12 cc.

Sealing pin 94 is preferably made of a type of polymer such as polypropylene, and it may be manufactured by an injection molding process. The diameter of the sealing pin 94 is generally in the range of 2-5 mm, preferably about 3 mm, and the diameter of its base is generally in the range of 5-12 mm, preferably about 6 mm. Pressure transfer element 93 is preferably made of a type of polymer such as polypropylene, and it may be manufactured by an injection molding process. The outer diameter of pressure transfer element 93 is generally in the range of 15-40 mm, preferably about 25 mm, and the inner diameter of the passage provided thereinside is generally in the range of 12-35 mm, preferably about 20 mm.

The length of contra elements 103 is generally in the range of 400-800 mm, preferably about 700 mm, their widths are generally in the range of 40-80 mm, preferably about 60 mm, and they may be manufactured from any material suitable for this purpose (e.g., leather, plastic, rubber). Pressure member 105 may be manufactured from any material suitable for this purpose (e.g., rubber) and may be inflated to volume ranges of 100-600 cc. Female fitting 102 may be manufactured by an injection molding process from a type of polymer such as polypropylene its outer diameter is generally in the range of 20-60 mm, preferably about 30 mm, its inner diameter is determined according to the diameter of male fitting 92 to allow a sealed connection therebetween, and the diameter of opening 101 may be determined according to the inner diameter of the passage of pressure transfer element 93, for instance about 20 mm. Fastening means 104 may be implemented by a cord lock clip buckle or Velcro® strips for instance.

FIGS. 12-13 demonstrate application of the self-regulating tourniquet of the invention to specific pressure points of a human subject 120. In FIG. 12, the self-regulating tourniquet module 100 is specially adapted to apply direct pressure to a section of the torso. For this purpose two or more contra elements may be attached to the sides of tourniquet module 100, and fastened over torso sections. For example, one contra element 121 may be fastened over the a shoulder opposite to the location in which the tourniquet module 100 is applied, and another contra element 122 may be fastened around the torso as shown in FIG. 12.

FIG. 13 demonstrates application of tourniquet modules 100 of the invention to an arm and a leg of a human subject 120.

The tourniquet modules 100 can be attached or be part of a built-in system being part, for example, of uniforms, were the pressure member 105 is attached on the upper part of the limbs (arms and legs), thereby providing a quick installation and operation of the tourniquet without requiring application of the same around the limbs, an operation that will most probably require assistance in case of serious injuries.

The modular tourniquet 100 of the invention may be pressurized by a liquefied gas contained in pressure source 91 which may be embodied as a low pressure canister such as an aerosol, utilizing liquefied gases such as HFC (hydrofluoro carbon) like tetra fluoro ethane or compressed gas such as Nitrogen or Carbon Dioxide or air. Alternatively, the modular tourniquet 100 of the invention may be pressurized by a pump, electric or mechanical pump for building the required pressure.

The pressure regulation may be performed utilizing mechanical means like an over pressure valve as shown in FIG. 1 or by electronic sensing as used in the portable self administered electronic blood pressure devices known in the art. These devices measure the turbulent flow of the blood by utilizing sensors that are attached to the skin of the subject, thus they are capable of diagnosing the blockage of the blood flow via a limb. These devices may be programmed to detect when there is no blood flow via an organ and for generating respective control signals for keeping the tourniquet pressure within a corresponding pressure range to maintain blood flow blockage. This will allow a control unit to apply the absolute minimum pressure required to stop the turbulent flow. In case wherein blockage of the laminar blood flow is required, a somewhat higher pressure rage may be needed, which may be easily achieved by suitable programming of the control unit.

The pressure unit can be either static part or otherwise be attached when needed. These preferred embodiment of the invention would shorten the time that is needed to install the tourniquet unit and will allow for the single wounded man to operate it by himself.

The same unit when using the automated blood pressure monitoring device can be made to operate on a remote command or by sensing the drop of blood pressure to a certain level or by measuring the changes in electro conductivity of the skin or by other similar means.

FIG. 14 demonstrates a possible embodiment utilizing a control unit 145 for regulating the pressure applied by the tourniquet modules 100. In this preferred embodiment the inflation of the pressure member 105 of the tourniquet modules 100 is controlled via suitable means such as controllable valves (not shown), which are linked to controller 145 via conducting wires 146. In this preferred embodiment of the invention the tourniquet modules 100 may be embedded in wearable articles such as uniforms. Pressure module 90 may be attached to each tourniquet module 100 separately or to a central point operable for applying pressure to each of the tourniquet modules 100 via controllable valves (not shown). Blood pressure sensors 140 are optionally embedded in the uniform below the location of each tourniquet module 100, where said sensors are also electrically connected to controller 145 via conducting wires 146. When embodied in uniforms for example, wires 146 may pass within the uniform may be stitched thereto.

When the blood pressure monitoring is carried out by electronic means as done in the different automatic blood pressure measuring devices, the electronic data can be automatically or on request, transmitted to a central command of the unit allowing each commander to know the physiological state of its troops. The whole system can be made modular with a changeable and removable pressure unit or each module can contain its own pressure unit.

It will be appreciated by persons skilled in the art that the scope of the present invention is not limited by what has been shown and described hereinabove, merely by way of example. Rather, the scope of the present invention is limited solely by the claims, which follow:

1. A self-regulating tourniquet which includes:
a pressure source;
pressure applicator apparatus associated with said pressure source and operative to apply pressure therefrom to a subject limb; and
a pressure regulator associated with said pressure source and said pressure member, operative to restrict the pressure transferred from said pressure source to the limb to a designated maximum pressure applied to the subject limb.

2. A self-regulating tourniquet according to claim 1, wherein said pressure regulator is configured to limit the pressure transferred from said pressure source to the limb to a predetermined pressure applied to the subject limb.

3. A self-regulating tourniquet according to claim 1, wherein said pressure regulator includes apparatus for adjusting the maximum pressure applied to the subject limb.

4. A self-regulating tourniquet according to claim 1, wherein said pressure applicator apparatus includes:
a pressure member, for directly applying pressure to a selected area on the subject limb; and
a contra member, adapted to at least partially circumvent the subject limb, and operative to resistively cooperate with said pressure member in applying a pressure to the subject limb.

5. A self-regulating tourniquet according to claim 4, wherein said pressure member is an inflatable member, and said pressure source is a portable fluid pressure source.

6. A self-regulating tourniquet according to claim 4, wherein said pressure source is a manually operable source by which a tension force may be applied to said contra member, and said pressure regulator includes apparatus for locking said contra member when the pressure applied to the subject limb reaches the maximum pressure.

7. A self-regulating tourniquet which includes:
a pressure source of liquified gas; pressure applicator apparatus associated with said pressure source and operative to apply pressure therefrom to a subject limb; and
liquified gas acting as a pressure regulator, associated with said pressure source and said pressure member, operative to regulate the pressure transferred to the limb to a maximum pressure of the steady-state of the liquid/gas phases.

8. A self-regulating tourniquet according to claim 6, wherein said liquified gas is selected to produce a steady-state pressure of a predetermined pressure applied to the subject limb at a given, working temperature.

9. A self-regulating tourniquet according to claim 6, wherein the temperature of said liquified gas is controlled by the body heat of the subject to produce a more stable steady-state pressure.

10. A self-regulating tourniquet according to claim 7, wherein the pressure source and the pressure applicator apparatus are separate modules adapted to be engaged by connecting means and thereby establish gas passage from the pressure source to said pressure applicator.

11. A self-regulating tourniquet according to claim 10, wherein a plurality of pressure applicators are embedded in wearable articles so as to at least partially circumvent limbs of a subject and operative to apply a pressure to the subject limb when engaged with a pressure source.
